(19) United States
(12) Patent Application Publication
(10) Pub. No.: US 2008/0293815 A1
(43) Pub. Date: Nov. 27, 2008

(54) HCA FOR IMPROVED HDL SERUM LIPIDS PROFILE

(75) Inventor: Dallas L. Clouatre, Berkeley, CA (US)

Correspondence Address: FOLEY & LARDNER LLP, 111 HUNTINGTON AVENUE, 26TH FLOOR, BOSTON, MA, 02199-7610, US

(73) Assignee: Glykon Technologies Group, LLC

(21) Appl. No.: 11/873,110

(22) Filed: Oct. 16, 2007

Related U.S. Application Data
(60) Provisional application No. 60/852,562, filed on Oct. 18, 2006.

(51) Int. Cl. A61K 31/194; A61P 3/00

(52) U.S. Cl. 514/574

(57) ABSTRACT

Described herein are compositions and methods for increasing serum high-density cholesterol lipoprotein (HDL) levels in an individual in need thereof comprising orally administering an effective amount of HCA.

### CROSS-REFERENCE TO RELATED PATENT APPLICATIONS

This application claims the benefit of U.S. Provisional Patent Application No. 60/852,562, filed Oct. 18, 2006, which is incorporated herein by reference in its entirety.

### FIELD OF THE INVENTION

This invention relates to pharmaceutical compositions containing HCA, its salts and related compounds useful for improving serum levels of high-density lipoprotein cholesterol (HDL) and the ratio of high-density lipoprotein cholesterol to low-density lipoprotein cholesterol (LDL) and total cholesterol, including in the face of a blood serum cholesterol raising diet.

### BACKGROUND OF THE INVENTION

Cardiovascular diseases as a category remain the single largest cause of death in the United States and most other industrialized countries, with serum cholesterol being singled out most often for medical treatment. Cholesterol, as a fatty substance, is not water-soluble, hence it is not soluble in the serum or fluid portion of the blood. Various names are attached to cholesterol depending upon the protein packets (lipoproteins) in which it is encased for transport. LDL cholesterol refers to cholesterol carried by low-density lipoproteins, which are relatively large particles. Larger still are the VLDL (very low-density lipoprotein) particles. In contrast with these, the HDL or high-density lipoprotein particles are relatively small. As a rule, the VLDL and LDL packets carry cholesterol from the liver and the storage areas via the blood to organs and other tissues. HDL cholesterol performs just the reverse action—it carries cholesterol back to the liver for disposal via the bile.

In common parlance, LDL cholesterol is referred to as the “bad” cholesterol, whereas HDL is termed the “good” cholesterol. However, the roles of various cholesterol fractions in cardiovascular diseases presently are hotly contested in the medical research literature. Moreover, it is not unusual to differentiate at least 6 types of hyperlipidemia through analyses of the type(s) of serum lipids that are involved and whether the condition is primary (usually meaning inherited) or secondary. Of all the cholesterol fractions, arguably it is the HDL components that over the last decade have received the greatest validation as being important in maintaining healthy cardiovascular functioning.

Relatively few compounds have shown a strong impact on increasing HDL or improving the ratio of HDL to LDL and to total cholesterol. Serum lipids lowering drugs, such the cholesterol fractions, arguably it is the HDL components that over the last decade have received the greatest validation as being important in maintaining healthy cardiovascular functioning.

Relatively few compounds have shown a strong impact on increasing HDL or improving the ratio of HDL to LDL and to total cholesterol. Serum lipids lowering drugs such as the statins even in the instances in which their effect reaches statistical significance in large cohorts, have not been shown to offer more than minor improvements in this area—changes almost always less than or on the order of those found with alterations in diet and/or exercise patterns. Some natural compounds, such as niacin and garlic, improve HDL levels and ratios, but niacin can lead to liver toxicity at therapeutic levels of intake and garlic often is not well tolerated at the dosages required for benefits. Of new drugs offered, it is the peroxisome proliferator activated receptor (PPAR) modulators that have shown the most promise. Nevertheless, these, too, have many drawbacks, including weight gain.

Previous work has not shown any direct benefit of Hydroxycitric Acid (abbreviated herein as HCA) or its salts in the area of HDL regulation. Indeed, and very surprisingly, researchers in only a handful of papers or other reports appear even to have looked at HDL levels in experiments based upon HCA as an active ingredient. That this would be the case with regard to early work on the compound undertaken in the 1980's is perhaps understandable inasmuch as HDL was neither widely tested nor its health role appreciated until the last ten years. However, as will be shown below, even in late 2006 it remains the case that only the current invention has recognized the great benefit of HCA in increasing HDL cholesterol and improving the ratio of HDL to LDL cholesterol and to total cholesterol.

All previous work has shown either no benefit under such conditions or results attributable to other ingredients or to exercise alone. Thus, there is a need to overcome to positively effect serum high-density lipoprotein levels.

### SUMMARY OF THE INVENTION

In a startling result, the present inventor has demonstrated that properly prepared HCA salts used by themselves and not as part of some program or in conjunction with other supplements to the diet dramatically increase HDL levels. This invention relates to pharmaceutical compositions containing HCA, its salts and related compounds useful for improving serum levels of high-density lipoprotein cholesterol (HDL) and the ratio of high-density lipoprotein cholesterol to low-density lipoprotein cholesterol (LDL) and total cholesterol, including in the face of a blood serum cholesterol raising diet. The invention provides that supplementation with (−) HCA, its salts and related compounds are useful for increasing HDL levels and for improving not only HDL levels, but also for inhibiting the deleterious effects of a cholesterol challenge on the HDL:LDL ratio. This finding offers benefits against a range of cardiovascular diseases. These benefits of HCA are especially pronounced with the use of the preferred salts of the acid, potassium HCA and potassium-magnesium HCA, and can be further potentiated by the use of a controlled-release form of the compound.

In one embodiment, the invention is directed to a method for increasing serum high-density cholesterol lipoprotein (HDL) levels in an individual in need thereof, comprising orally administering an effective amount of HCA. In a particular embodiment, HCA is administered in a therapeutically-effective amount in its free acid or lactone form. In another embodiment, HCA is supplied in a therapeutically-effective amount of the alkali metal salts potassium or sodium (−) HCA. In another embodiment, HCA is supplied in a therapeutically-effective amount of the alkaline earth metal salts calcium or magnesium (−) HCA. In yet another embodiment, HCA is supplied in a therapeutically-effective amount of a mixture the alkali metal salts and/or the alkaline earth metal salts of (−) HCA or some mixture of alkali metal salts and alkaline earth metal salts of (−) HCA or in the form of therapeutically effective amide and/or ester derivatives of HCA. In another embodiment, HCA is supplied in a therapeutically-effective amount as the free acid, lactone or as one or more of the salts or other derivatives of the free acid and is delivered in a controlled release form.

In another embodiment, the present invention is directed to a method for increasing the HDL:LDL ratio in a subject, comprising administering a therapeutically effective amount of a composition that is a salt of HCA and a suitable alkaline or alkali earth metal. In a particular embodiment, the alkaline earth metal is calcium or magnesium. In a particular embodiment, the alkali earth metal is potassium or sodium. In another embodiment, the composition allows for a delivery dose of between about 750 mg and 10 grams. In yet another embodiment, the composition is part of a controlled-release formulation.

### DETAILED DESCRIPTION

The discovery that HCA improves HDL levels and improves the HDL to LDL ratio in the face of dietary cholesterol challenge allows for the creation of novel and more efficacious approaches to preventing and ameliorating cardiovascular diseases. Furthermore, this discovery makes possible the development of adjuvant modalities that can be used to improve the results realized with other treatment compounds while at the same time reducing the side effects normally found with such drugs.

HCA delivered in the form of its potassium salt is efficacious at a daily dosage (bid or tid) of between 750 mg and 10 grams, preferably at a dosage of between 3 and 6 grams, between 1 and 7 grams, between 2 and 5 grams, and between 2.5 and 4 grams for most individuals. A daily dosage above 10 grams is desirable under some circumstances, such as with extremely large or resistant individuals.

It is an object of the present invention to provide a method for preventing treating or ameliorating conditions that involve cardiovascular components related to low HDL levels and poor HDL:LDL ratios due to poor dietary practices. As used herein ameliorating conditions that involve low HDL:LDL ratios means increasing the HDL:LDL ratio, for example, by increasing the level of HDL while lowering or keeping the LDL level constant. As used herein, the levels of these lipoproteins can be determined in, for example, serum samples obtained from a patient.

Very few compounds are known that have any reliable effect in these areas and these compounds typically are associated with a variety of side effects. For instance, other PPAR-□ modifiers cause weight gain and statin drugs, which are weak as inhibitors of calcification, are noted for such numerous and unpleasant side effects that approximately seventy-five percent of patients discontinue use within two years. As disclosed herein, one of skill in the art can now using therapeutically-effective amounts of compositions of HCA, including especially through controlled-release formulations, as an adjuvant to cardiovascular drugs and other drugs.

As used herein, “controlled-release” refers to a formulation designed to consistently release a predetermined, therapeutically effective amount of drug or other active agent such as a polypeptide or a synthetic compound over an extended period of time, with the result being a reduction in the number of treatments necessary to achieve the desired therapeutic effect. In the matter of the present invention, a controlled-release formulation would decrease the number of treatments necessary to achieve the desired effect. The controlled-release formulations of the present invention achieve a desired pharmacokinetic profile in a subject, preferably commencement of the release of the active agent substantially immediately after placement in a delivery environment, followed by consistent, sustained, preferably zero-order or near zero-order release of the active agent.

The free acid form and various salts of HCA (calcium, magnesium, potassium, sodium and mixtures of these) are available commercially and useful in the methods of the present invention. Not all HCA forms are equally potent and compounds are generally useful in this descending order of efficacy: potassium salt, sodium salt, free acid, magnesium salt, and calcium salt. Exact dosing will depend upon the form of HCA used, the weight of the individual involved, and the other components of the diet. Controlled-release can also be expected to improve results by aiding in maintaining a sustained exposure to the drug as required for therapy. The previously patented HCA derivatives (mostly amides and esters of hydroxycitric acid, the patents for which are now expired, to with, U.S. Pat. Nos. 3,993,668; 3,919,254; and 3,767,678) likely are roughly equivalent to the HCA sodium salt in efficacy.

The compositions and methods described herein are for individuals, e.g., patients in need of increased levels of serum HDL. As used herein, “patient” and “subject” mean all animals including humans. Examples of patients or subjects include humans, cows, dogs, cats, goats, rabbits, sheep, and pigs.

As used herein, “treatment” refers to the administration of medicine or the performance of medical procedures with respect to a patient, for either prophylaxis (prevention) or to cure the infirmity or malady in the instance where the patient is afflicted.

A “therapeutically effective amount” is an amount sufficient to decrease, prevent, or ameliorate the symptoms associated with a medical condition. In the context of hormonal therapy it can also mean to normalize body functions or hormone levels in disease or disorders.

All publications mentioned herein are incorporated by reference to the extent they support the present invention. This invention and embodiments illustrating the method and materials used may be further understood by reference to the following non-limiting examples.

### Exemplification

### Introduction

HCA is a naturally-occurring substance found chiefly in fruits of the species of Garcinia, and several synthetic derivatives of citric acid have been investigated extensively in regard to their ability to inhibit the production of fatty acids from carbohydrates, to suppress appetite, and to inhibit weight gain. (Sullivan A C, Triscari J. Metabolic regulation as a control for lipid disorders. I. Influence of (−)-hydroxycitrate on experimentally induced obesity in the rodent. American Journal of Clinical Nutrition 1977; 30:767-775). Weight loss benefits were first ascribed to HCA, its salts and its lactone in U.S. Pat. No. 3,764,692 granted to John M. Lowenstein in 1973. The claimed mechanisms of action for HCA, most of which were originally put forth by researchers at the pharmaceutical firm of Hoffmann-La Roche, have been summarized in at least two U.S. patents. In U.S. Pat. No. 5,626,849 these mechanisms are given as follows: “(−) HCA reduces the conversion of carbohydrate calories into fats. It does this by inhibiting the actions of ATP-citrate lyase, the enzyme that converts citrate into fatty acids and cholesterol in the primary pathway of fat synthesis in the body. The actions of (−) HCA increase the production and storage of glycogen (which is found in the liver, small intestine and muscles of mammals) while reducing both appetite and weight gain. (−) HCA also causes calories to be burned in an energy cycle similar to thermogenesis . . . (−) HCA also increases the clearance of LDL cholesterol . . . ” U.S. Pat. No. 5,783,603 further argues that HCA serves to disinhibit the metabolic breakdown and oxidation of stored fat for fuel via its effects upon the compound malonyl CoA and that gluconeogenesis takes place as a result of this action. The position that HCA acts to unleash fatty acid oxidation by negating the effects of malonyl CoA with gluconeogenesis as a consequence (McCarty M F. Promotion of hepatic lipid oxidation and gluconeogenesis as a strategy for appetite control. Medical Hypotheses 1994; 42:215-225) is maintained in U.S. Pat. No. 5,914,326.

Most of the primary research on HCA was carried out by Hoffman-La Roche nearly three decades ago. The conclusion of the Roche researchers was that “no significant differences in plasma levels of glucose, insulin, or free fatty acids were detected in (−) HCA-treated rats relative to controls. These data suggest that peripheral metabolism, defined in the present context as metabolite flux, may be involved in appetite regulation . . . ” (Sullivan, Ann C. and Joseph Triscari. Possible interrelationship between, metabolite flux and appetite. In D. Novin, W. Wyriwicka and G. Bray, eds., Hunger: Basic Mechanisms and Clinical Implications (New York: Raven Press, 1976) 115-125).

HCA is highly researched as of late 2006, with 169 citations appearing on PubMed under “hydroxycitrate” and 113 appearing under “HCA.” Again, and quite surprisingly, these reports do not appear to disclose a benefit from HCA ingestion to improve blood levels of high-density lipoprotein cholesterol (HDL) or to improve the ratio of HDL to low-density lipoprotein cholesterol (LDL) through an effect based primarily on increasing HDL as opposed to lowering LDL.

Already in 1990, it had been demonstrated that HCA can reduce LDL levels. (Berkhout T A, Havekes L M, Pearce N J, Groot P H. The effect of (−) HCA on the activity of the low-density-lipoprotein receptor and 3-hydroxy-3-methylglutaryl-CoA reductase levels in the human hepatoma cell line Hep G2. Biochem J. 1990 Nov. 15; 272(1):181-6). Such an effect on LDL and total cholesterol was expected based upon the mechanisms of action proposed by Roche in the 1980's and the large number of studies showing a reduction in de novo lipogenesis.

As is well established in the medical literature, however, very few of the pharmaceutical agents currently employed to regulate blood lipids show significant benefit with regard to improving HDL levels and there is seldom any relationship between lowering LDL cholesterol levels and raising HDL cholesterol levels. Indeed, the failure to significantly improve HDL routinely has been put forth as an explanation as to why a large number of drugs once used to treat dyslipidemia, e.g., dextrothyroxine and cholestyramine, did not improve cardiovascular mortality despite reducing total cholesterol, LDL and triglyceride levels. Tellingly, the first drug to be shown to reduce the rate of cardiovascular events, gemfibrozil, has only a minor impact on LDL, but significantly increases HDL. This was first shown approximately 15 years ago and confirmed again in 2006. (Otvos J D, Collins D, Freedman D S, Shalaurova I, Schaefer E J, McNamara J R, Bloomfield H E, Robins S J. Low-density lipoprotein and high-density lipoprotein particle subclasses predict coronary events and are favorably changed by gemfibrozil therapy in the Veterans Affairs High-Density Lipoprotein Intervention Trial. Circulation. 2006 Mar. 28; 113(12): 1556-63). In pharmaceutical treatment, there is no necessary relationship between reducing LDL and improving HDL. Unfortunately, the reduction of cardiovascular events often touted for cholesterol-lowering drugs seldom translates into a similar reduction in rates of mortality. Quite a number of widely prescribed drugs, such as clofibrate, actually increase total mortality. (Barter P J, Rye K A. Circulation. 2006 Mar. 28; 113(12):1553-5). The currently favored drugs for the treatment and prevention of cardiovascular disease, the statins, have demonstrated only minor improvements in HDL levels.

Surprisingly, only a total of three studies in almost forty years appear to have looked directly at the impact of HCA (as a single active ingredient) on HDL levels. A few other published studies based upon other experiments have indirectly addressed the issue of HDL, but all of these have included one or more compounds other than HCA and these other compounds in all cases are known to influence total cholesterol, LDL and HDL cholesterol levels. Some trials have included chitosan, chromium polynicotinate and thus cannot qualify as tests of the actions of HCA alone in the area of serum lipids. Typical of these studies is Daher C F, Koulajian K B, Haddad N, Baroody G M. Effect of hydroxycut intake on fasted and postprandial lipemia in rats. J Toxicol Environ Health A. 2006 September; 69(17):1587-601. In this case, not only HCA, but also L-carnitine, chromium picolinate and α-lipoic acid were included in the test arm. The HCA source was Super CitriMax™. The other three items each are claimed to exert at least some positive influence on HDL levels as well as to improve blood sugar regulation. Nevertheless, on a high fat diet the formulation actually dramatically increased blood sugar levels.

Mahendran and Devi conducted the early studies to test the impact of HCA on HDL cholesterol levels (Mahendran P. Devi C S, Effect of Garcinia cambogia extract on lipids and lipoprotein composition in dexamethasone administered rats. Indian J Physiol Pharmacol. 2001 July; 45(3):345-50). In this experiment, the administration of the glucocorticoid dexamethasone was shown to increase LDL and VLDL, but not to influence HDL. The particular preparation of HCA used in this experiment prevented the elevation of LDL and VLDL and thus undesirable changes in lipid profile upon dexamethasone administration. Nevertheless, the experiment did not demonstrate an impact upon HDL and the researchers did not claim such an effect.

A second trial, performed on human subjects in Japan using an HCA extract also failed to demonstrate a significant effect of HCA administration upon HDL levels in 16 weeks (Hayamizu K, Ishii Y. Kaneko I, Shen M, Okuhara Y. Shigematsu N. Tomi H. Furuse M, Yoshino G. MD, Shimasaki H. Effects of Garcinia cambogia (HCA) on visceral fat accumulation—a double-blind, randomized, placebo-controlled trial. Current Therapeutic Research 2003 September/October; 64, 8:551-567). An earlier and less definitive write-up of this study that reached the same conclusion was published in 2001.

The third trial, also a human study, led to the publication of at least five articles, all of which rely upon the same experimental data. This clinical test, which was conducted in India, has been presented by Preuss and coworkers (Preuss H G, Bagchi D, Bagchi M, Rao C V, Dey D K, Satyanarayana S. Effects of a natural extract of HCA (HCA-SX) and a combination of HCA-SX plus niacin-bound chromium and Gymnema sylvestre extract on weight loss. Diabetes Obes Metab. 2004 May; 6(3):171-80; see also, U.S. Pat. No. 7,119,110; Shara M, Ohia S E, Schmidt R E, Yasmin T, Zardetto-Smith A, Kincaid A, Bagchi M, Chatterjee A, Bagchi D, Stobs S J. Physico-chemical properties of a novel (−) HCA extract and its effect on body weight, selected organ weights, hepatic lipid peroxidation and DNA fragmentation, hematology and clinical chemistry, and histopathological changes over a period of 90 days (Mol Cell Biochem. 2004 May; 260(1-2):171-86). These human studies did not demonstrate a significant effect of HCA upon HDL cholesterol levels using the potassium-calcium HCA salt known as Super CitriMax® (HCA-SX). The lack of effect of the particular HCA preparation used is explicit in the data found in Shara M, et al. (2004).

The HCA-SX clinical study (Preuss H G, et al., Effects of a natural extract of HCA (HCA-SX) and a combination of HCA-SX plus niacin-bound chromium and Gymnema sylvestre extract on weight loss (Diabetes Obes Metab. 2004 May; 6(3):171-80) recruited obese subjects who were placed on boxed meals and supervised exercise program (30 minutes per day 5 days per week) for 8 weeks. The placebo was not characterized and the treatment groups did not appear to be randomized. Initial body weight in kilograms (BW) and body mass index (BMI) for the arms are as follows: Placebo (80.44±2.36 (BW) and 32.49±0.57 (BMI)); HCA-SX (91.74±3.50 (BW) and 34.71±1.22 (BMI)); HCA-SX formula* (92.41±3.84 (BW) and 37.33±1.70 (BMI)). *HCA-SX formula included chromium polynicotinate and Gymnema sylvestre extract.

Under the conditions described, at the end of 8 weeks the HCA-SX group, which started out weighing 24.86 pounds more than did the Placebo group, showed a small 7.89% improvement in HDL, which just reached significance, whereas there was no significant change in Placebo. This small improvement in HDL level, however, was not likely due to dietary intervention with the supplement HCA-SX, but rather to diet and exercise alone. It is well established that exercise increases HDL based upon BMI, which is to say, exercise is known to improve serum HDL and this effect increases in direct proportion to the initial BMI. The effect of exercise may be attenuated in the non-obese, but becomes more pronounced at BMI increases. (Kondo T. Kobayashi I, Murakami M. “Effect of exercise on circulating adipokine levels in obese young women.” Endocr J. 2006 April; 53(2): 189-95).

Note that the HCA-SX group is recorded as initially being far more obese than is Placebo and the HCA-SX formula group, even more so. Exercise alone is quite sufficient to yield an effect of the magnitude reported by Preuss, et al., not only on HDL, but also on the HDL/cholesterol ratio and in the area of leptin. It was not clear that the “placebo” used in these studies was actually a sugar, hence not a true placebo at all. In Placebo after eight weeks of boxed meals and supervised exercise, LDL, total cholesterol and triglycerides all increased, whereas HDL declined (although no changes reached significance)—exactly the opposite finding from virtually all other published studies conducted according to the methods described in this trial. Very simply, walking reduces LDL-C and the TC/HDL-C ratio in adults independent of changes in body composition even in the absence of caloric restriction. (Kelley G A, Kelley K S, Tran Z V, Walking and Non-HDL-C in adults: a meta-analysis of randomized controlled trials. Prev Cardiol. 2005 Spring; 8(2):102-7).

This lack of demonstrated benefit in the area of HDL by the HCA-SX (Super CitriMax®) version of potassium-calcium HCA is confirmed by the earlier animal study relied upon by U.S. Pat. No. 7,119,110 and which utilized the same HCA salt. This study (Shara M, et al. Physico-chemical properties of a novel (−) HCA extract and its effect on body weight, selected organ weights, hepatic lipid peroxidation and DNA fragmentation, hematology and clinical chemistry, and histopathological changes over a period of 90 days (Mol Cell Biochem. 2004 May; 260(1-2):171-86) reported no changes in serum cholesterol readings or in any clinical chemistry finding at 30, 60 or 90 days compared with control even when HCA-SX constituted 5% of the diet. The paper's authors (who include Bagchi, also listed as an inventor on U.S. Pat. No. 7,119,110) maintained that 0.2% HCA in the diet is equivalent to the recommended dosage for human intake, hence equivalent to the amount used in the Preuss paper cited above. Weight reduction versus control as shown in this trial was not robust, and did not even approach the weight reduction that Roche reported under similar test conditions using a synthesized trisodium HCA. As a final note, U.S. Pat. No. 7,119,110—covering insulin resistance syndrome—does not make any claims regarding HCA for HDL improvements. Indeed, this patent art argues that its primary substance, chromium polynicotinate, offers benefits in insulin resistance syndrome only in combination with three other ingredients, one of which can, but need not be, HCA.

Aside from the foregoing, U.S. Pat. No. 6,221,901 (Magnesium HCA, method of preparation, applications, and compositions in particular pharmaceutical containing same) touches on the issue of HCA and HDL cholesterol in one of its examples and therefore might be said to be relevant. However, this patent discusses HCA only as a anion to magnesium as a cation, a point that is clear from the fact that neither HCA nor other forms of HCA are mentioned except magnesium HCA and from other aspects of the patent's language. For instance, as a type of HCA salt, magnesium HCA was first proposed by Lowenstein in 1973 (U.S. Pat. No. 3,764,692). Moreover, the small (8.5%) increase in HDL shown in an animal model with the particular magnesium salt produced according to the teachings of this patent is typical of results found with magnesium supplementation (Rosanoff A Seelig M S, Comparison of mechanism and functional effects of magnesium and statin pharmaceuticals, J Am Coll Nutr. 2004 October; 23(5):501S-505S). Indeed, all of the benefits reported in the examples of this patent are known from long existing literature covering the benefits of magnesium. The primary issue faced with magnesium supplementation is the difficulty in repletion given that most magnesium salts lead to osmotic laxative effects, and this difficulty would seem to be the primary point of this patent's art. Notably, there are no claims made in this patent regarding HCA nor for HCAs other than magnesium. Similarly, there are no specific claims made regarding benefits in the area of HDL levels.

One issue of significance that partially explains the surprising failure of prior art to demonstrate the benefits of HCA with regard to HDL metabolism is the nature of the HCA preparations tested. As already observed, the findings with Super CitriMax® with regard to weight regulation do not approach those reported by Roche using a pure trisodium HCA. Compared to the various Roche-sponsored studies, Super CitriMax® gave very poor results as reported for food intake and body weight changes in Shara M, Ohia S E, Yasmin T, Zardetto-Smith A, Kincaid A, Bagchi M, Chatterjee A, Bagchi D, Stohs S J. Dose- and time-dependent effects of a novel HCA extract on body weight, hepatic and testicular lipid peroxidation, DNA fragmentation and histopathological data over a period of 90 days (Mol Cell Biochem. 2003 December; 254(1-2):339-46).

Recent studies both in Europe and in the US specifically have noted that there are differences in HCA preparations depending upon production techniques and other factors. (Louter-van de Haar J. Wielinga P Y, Scheurink A J, Nieuwenhuizen A G. Comparison of the effects of three different (−) HCA preparations on food intake in rats. Nutr Metab (Lond). 2005 Sep. 13; 2:23). (Wielinga P Y, Wachters-Hagedoorn R E, Bouter B, van Dijk T H, Stellaard F, Nieuwenhuizen A G, Verkade H J, Scheurink A J. HCA delays intestinal glucose absorption in rats. Am J Physiol Gastrointest Liver Physiol. 2005 June; 288(6):G1144-9). Likewise, a trial performed at Georgetown University Medical School in the laboratory of Dr. Harry Preuss, demonstrated that the potassium-calcium HCA tested presented no benefits at physiologic doses with regard to insulin metabolism and blood pressure regulation, whereas both the particular potassium- and potassium-magnesium salts tested yielded significant results. (Clouatre, D, Preuss H, et al. Comparing Metabolic and Inflammatory Parameters Among Rats Consuming Different Forms of Hydroxycitrate. The American College of Nutrition Annual Convention. 2005; Abstr. #61). According to Shara M, et al. (2004) cited above, Super CitriMax® is not a pure HCA potassium-calcium salt. “A typical compositional analysis of Super CitriMax® contains 60% HCA in its free form, 1.0% HCA in its lactone form, 10% calcium, 15% potassium, 0.5% sodium, 0.05% total phytosterols, 0.3% total protein, 4.5% moisture and 8.5% soluble dietary fiber (by difference). Super CitriMax® also contains 0.1% magnesium, 0.03% iron, and trace amounts of manganese, copper, zinc, selenium, total fat and total sugar. Super CitriMax® provides approximately 150 calories per 100 g.” Similar quality control defects are characteristic of the other currently commercially available HCA salt.

### EXAMPLE 1

### Preparation of a Potassium-Magnesium Hydroxycitrate Salt

HCA is present to the extent of 20-30% in the dried fruit rinds of members of the Garcinia family, such as cambogia, atroviridis and indica. Water or water/alcohol extraction is sufficient to concentrate the acid as a lactone/acid mixture. Neutralization of the concentrated extract with KOH or MgO to a neutral pH and to stoichiometrically determined proportions can be achieved by methods known in the art (Y. S. Lewis, “Isolation and properties of hydroxycitric acid,” in Methods in Enzymology 13 (1969) 613-19. John M. Lowenstein in U.S. Pat. No. 3,764,692 similarly alludes to the known methods of creating salts of HCA). To create a potassium/magnesium salt appropriate to the invention, 100 ml of a 5% by weight HCA solution in water (0.0227 M) is neutralized to pH 7 with 38 ml of a 10% by weight KOH solution in water (component A). Twenty milliliters (20 ml) of a 5% HCA solution in water is mixed with 0.27 g of MgO and allowed to stand for 30 minutes (component B). Components A and B are mixed to give about 158 ml of the K—Mg—HCA solution. It is important that protein, gum, pectin and/or fiber not remain in the finished product and that the HCA be fully reacted with its cation(s).

### EXAMPLE 2

### Preparation of a Potassium HCA Salt

Washed Garcinia fruits were pitted and cut into small pieces. A mixture of 1.5 kg fruit flesh and 1.5 L of water were crushed using a blender. The slurry was filtered using a piece of cloth and Ca(OH)₂ powder was added to the filtrate in order to clarify the juice. The clarified juice (pH 3.5) was filtered again using filter paper. The clear juice was heated and reacted with 10% KCO₃ solution until pH of 9 was reached. The mixture was concentrated using a rotary evaporator at a temperature lower than 50° C. to remove most of the water. To prepare the 0.5% KHCA solution used in Example 3, 0.5 ml extract was dissolved to yield 100 ml solution. The 1% and 2% solutions were prepared similarly.

### EXAMPLE 3

### Animal Trial Using HCA to Improve HDL Levels

Twenty male rats of Wistar strain were provided (weight about 200 g each, 1.5 months old. The animals were fed a standard laboratory chow ad libitum. After six weeks of adaptation, the rats were divided into four groups, five rats in each group. All rats were given cholesterol diets for ten days. Table 1 shows the composition of the feed.

TABLE 1
Composition of feed given to the rats

| 1. Materials | 2. Weight | |
|---|---|---|
| Husk/pollard | 9.37 | kg |
| Corn meal | 6.87 | kg |
| Soy cake | 2.5 | kg |
| Fish meal | 3.0 | kg |
| Bone meal | 1.75 | kg |
| Barco - Vegetable Oil | 0.25 | kg |
| NDCP | 1.125 | kg |
| Premix | 0.031 | g |
| Salt | 0.125 | g |
| Molasses | 0.87 | g |
| Lysine | 0.05 | g |
| Methionine | 0.05 | g |
| Cholesterol** | 31.50 | g |

**Given after 6 weeks of adaptation period

During the adaptation period, health was monitored by weighing and observing the animals. Healthy rats were indicated by increasing weight and did not show any health disorder. After 10 days of cholesterol-containing feed, all animals were in a state of hypercholesterolemia. One group served as the control, whereas group II, III, and IV received 0.5%, 1%, and 2% of KHCA (produced via the method of Example 2), respectively, in addition to the cholesterol diet. The salt solutions were given orally in 2 ml water daily. Group I received 2 ml of distilled water per 200 g body weight; group II received 1.03 mg KHCA in 2 ml water per 200 body weight; group III 2.06 mg; and group IV 4.12 mg. These treatments were continued for 2 weeks.

### Blood Serum Analysis

The animals were fasted for 12 hours prior to blood withdrawal. Rats were weighed and the 3-4 ml blood was obtained by amputating 2-3 cm of the tail. The blood was collected, let stand at room temperature for 30 minutes, and then centrifuged for 10-15 minutes. The serum was separated and refrigerated at 4° C. until ready for cholesterol analysis. Cholesterol level of the serum was analyzed using diagnostic kit (Boehringer Mannheim). Total cholesterol, LDL, and HDL were determined through spectrophotometric instrument (Hitachi). These are protocols frequently used in clinical laboratories.

### Data Analysis

Student's T-test was used to evaluate whether there was a difference between the control and each of the treated groups by comparing the data before and after the treatment. One-way variance analysis was performed to see if there was a statistically significant difference among the groups.

### Results

All animals were in good condition. The average of body weights before hypercholesterolemia condition, at hypercholesterolemia condition, and after two weeks treatments are presented in Table 2.

TABLE 2
Body weight (in grams) at the start, at hypercholesterolemia,
and after treatment

| Group | | | |
|---|---|---|---|
| % solution/ | | | |
| KHCA mg | Start of | | End of |
| per day | Experiment | At Hypercholesterolemia | Experiment |
| Control | 161.7 | 200.8 | 217.0 |
| 0.5% KHCA | 162.8 | 202.4 | 199.5 |
| (1.03 mg) | | | |
| 1.0% KHCA | 157.7 | 195.0 | 193.2 |
| (2.06 mg) | | | |
| 2.0% KHCA | 166.9 | 192.7 | 183.7 |
| (4.12 mg) | | | |

Cholesterol concentrations in rat serum before hypercholesterolemia, at hypercholesterolemia, and after K—HCA treatment are shown in Table 3 and Tables 4A & 4B.

TABLE 3
The profile of total cholesterol, HDL and LDL as
affected by treatments.

| | | | | Hyper- | | | | | |
|---|---|---|---|---|---|---|---|---|---|
| | Start | | | cholesterolemia | | | End | | |
| | Total | HDL | LDL | Total | HDL | LDL | Total | HDL | LDL |
| Control | 72 | 41 | 30 | 81 | 37 | 67 | 99 | 35 | 103 |
| | 72 | 43 | 31 | 84 | 40 | 65 | 94 | 32 | 97 |
| | 70 | 39 | 26 | 79 | 31 | 77 | 91 | 31 | 95 |
| | 77 | 40 | 33 | 92 | 30 | 68 | 98 | 28 | 101 |
| | 75 | 36 | 33 | 80 | 29 | 63 | 96 | 29 | 77 |
| Average | 73.2 | 39.8 | 30.6 | 80.8 | 33.4 | 68 | 95.6 | 31 | 94.6 |
| 0.5% | 74 | 37 | 31 | 83 | 27 | 60 | 82 | 40 | 59 |
| KHCA | 73 | 40 | 28 | 84 | 28 | 70 | 80 | 59 | 77 |
| | 69 | 41 | 33 | 76 | 39 | 67 | 76 | 43 | 71 |
| | 72 | 39 | 27 | 80 | 31 | 59 | 79 | 53 | 60 |
| | 75 | 38 | 25 | 82 | 30 | 60 | 79 | 51 | 56 |
| Average | 72.6 | 39 | 28.8 | 81 | 31 | 63.2 | 79.2 | 49.2 | 64.6 |
| 1.0% | 69 | 36 | 28 | 75 | 28 | 67 | 73 | 58 | 69 |
| KHCA | 74 | 40 | 29 | 82 | 37 | 59 | 71 | 60 | 51 |
| | 72 | 43 | 33 | 84 | 37 | 54 | 75 | 49 | 48 |
| | 75 | 39 | 30 | 80 | 30 | 60 | 69 | 46 | 60 |
| | 73 | 37 | 25 | 77 | 27 | 62 | 67 | 52 | 63 |
| Average | 72.6 | 39 | 29 | 79.6 | 31.8 | 60.4 | 71 | 53 | 58.2 |
| 2.0% | 75 | 38 | 31 | 83 | 31 | 69 | 59 | 64 | 61 |
| KHCA | 70 | 40 | 29 | 85 | 37 | 61 | 66 | 61 | 46 |
| | 72 | 41 | 26 | 81 | 40 | 59 | 52 | 67 | 50 |
| | 73 | 36 | 33 | 79 | 33 | 60 | 58 | 59 | 44 |
| | 77 | 39 | 30 | 82 | 32 | 67 | 71 | 66 | 49 |
| Average | 73.4 | 38.8 | 29.8 | 82 | 34.6 | 63.2 | 61.2 | 63.4 | 50 |

TABLE 4A
The profile of total cholesterol, HDL and LDL as affected by treatments.

| | | | | HDL | | | LDL | | |
|---|---|---|---|---|---|---|---|---|---|
| | | | | | Hyper- | | | Hyper- | |
| | Total cholesterol | | | | cholesterolemia | End | | cholesterolemia | End |
| | | Hyper- | | | (Day | (Day | | (Day | (Day |
| | Start | cholesterolemia | End | Start | 10) | 24) | Start | 10) | 24) |
| Control | 73.2 | 80.8 | 95.6 | 39.8 | 33.4 | 31 | 30.6 | 68 | 94.6 |
| KHCA | 72.6 | 81 | 79.2 | 39 | 31 | 49.2 | 28.8 | 63.2 | 64.6 |
| 0.5% | | | | | | | | | |
| KHCA | 72.6 | 79.6 | 71 | 39 | 31.8 | 53 | 29 | 60.4 | 58.2 |
| 1.0% | | | | | | | | | |
| KHCA | 73.4 | 82 | 61.2 | 38.8 | 34.6 | 63.4 | 29.8 | 63.2 | 50 |
| 2.0% | | | | | | | | | |

TABLE 4B
The profile of total cholesterol/HDL and LDL/HDL Ratios as affected by
treatments.

| | Total Cholesterol/HDL | | | LDL/HDL Ratio | | |
|---|---|---|---|---|---|---|
| | | | End | | | End |
| | | Hyper-cholesterolemia | (Day | | Hyper-cholesterolemia | (Day |
| | Start | (Day 10) | 24) | Start | (Day 10) | 24) |
| Control | 1.84 | 2.41 | 3.08 | 0.77 | 2.04 | 3.05 |
| KHCA 0.5% | 1.86 | 2.61 | 1.61 | 0.74 | 2.04 | 1.31 |
| KHCA 1.0% | 1.86 | 2.50 | 1.34 | 0.74 | 1.9 | 1.1 |
| KHCA 2.0% | 1.89 | 2.36 | 0.97 | 0.77 | 1.83 | 0.79 |

### Discussion

After 10 days consumption of high-cholesterol diets, all rats reached a hypercholesterolemic condition (>80 mg/dL total cholesterol—considered high for rats).

In terms of total cholesterol reduction in comparison to the starting condition, there was a significant difference between treatment using 2.0% KHCA and those using 1.0%, as well as treatment using 0.5% K-HCA (the lowering of 12.2; 2.06, and 1.6 mg dL, respectively). In this experiment, therefore, the rats that received 4.12 mg KHCA daily showed the best result in the total cholesterol reduction.

A good indication also exhibited by the same group in terms of HDL (the “good” cholesterol) concentration in the blood serum. In this group, HDL concentration increased, whereas that of the control group decreased. The lowest level of LDL and the highest level of HDL were shown by rats receiving 2.0% (4.12 mg) KHCA daily.

Data showed that LDL concentrations of the treated rats at the end of experiment were still higher than those at the very start condition (before consuming high-cholesterol diet). There is a good prospect of LDL lowering, however, if compared to the control group, which kept increasing. The higher the salt concentration in the feeds the higher the reduction of the LDL concentration in the blood serum and the higher the HDL. The results show that 2 ml of 2 percent daily treatment of HCA for two weeks reduced serum cholesterol LDL levels from 63 mg/dl to 50 mg dl, increased HDL level from 35 to 63 mg/dl and reduced body weight.

There were another facts observed. KHCA administration resulted in lower feed consumption, but higher water consumption. The feces were softer than in the control group.

Weight reduction was also studied in this experiment (Table 2). Rats in the control group gain weight during the experiment. The KHCA-treated rats gained their weight until the hypercholesterolemic state and started to decrease after consuming the salt. Again, the higher the salt concentration given to the animal, the higher the weight reduction after the induction of the hypercholesterolemic state.

### EXAMPLE 4

### Preparation of a Standard Dosage Form

Numerous methods can be given as means of delivering HCA as required by the invention, including capsules, tablets, powders and liquid drinks. The following preparation will provide a stable and convenient dosage form as detailed in Table 5 below.

TABLE 5

| Ingredient | | Weight | Percent | 1 Kg Batch |
|---|---|---|---|---|
| 1. | Aqueous Potassium HCA | 500 gm | 62.5% | 0.63 |
| 2. | Calcium Carbonate | 50 gm | 6.25% | 0.06 |
| 3. | Potassium Carbonate | 50 gm | 6.25% | 0.06 |
| 4. | Anhydrous Lactose | 150 gm | 18.75% | 0.19 |
| 5. | Cellulose Acetate Pthalate | 50 gm | 6.25% | 0.06 |
| | Acetate | | | |

| | | | |
|---|---|---|---|
| Total | 800 gm | 100.00% | 100.00 |

A. Blend items 1-5 in mixing bowl until smooth and even.

B. Take the liquid and spray into spray-drying oven at 300° C. until white powder forms. When powder has formed, blend with suitable bulking agent, if necessary, and compress into 800 mg tablets with hardness of 10-15 kg. This will mean that each tablet, if starting with 62% KHCA polymer powder, will have about 31% KHCA. If the tablets are pressed to 1600 mg, however, the dose will be equal to 800×62% KHCA.

C. After pressing the granulate through the screen, make sure that it flows well and compress into oblong tablets.

D. Tablets should have a weight of 1600 mg and a hardness of 14+3 kg fracture force. When tablets are completed, check for disintegration in pH 6.8, 0.05M KH₂PO₄. Disintegration should occur slowly over 4-5 hours.

### EXAMPLE 5

### Preparation of an Enteric Softgel Dosage Form

Soft gelatin encapsulation is used for oral administration of drugs in liquid form. For this purpose, HCA can be provided in a liquid form by suspending it in oils, polyethylene glycol-400, other polyethylene glycols, poloxamers, glycol esters, and acetylated monoglycerides of various molecular weights adjusted such as to insure homogeneity of the capsule contents throughout the batch and to insure good flow characteristics of the liquid during encapsulation. The soft gelatin shell used to encapsulate the HCA suspension is formulated to impart enteric characteristics to the capsule to ensure that the capsule does not disintegrate until it has reached the small intestine. The basic ingredients of the shell are gelatin, one or more of the enteric materials listed above, plasticizer, and water. Care must be exercised in the case of softgels to use the less hygroscopic salts and forms of HCA or to pre-treat the more hygroscopic salts to reduce this characteristic. The carrier may need to be adjusted depending on the HCA salt, ester or amide used so as to avoid binding of the ingredients to the carrier. Water should not be used as a carrier. Various amounts of one or more plasticizer are added to obtain the desired degree of plasticity and to prevent the shell from becoming too brittle.

### EXAMPLE 6

Controlled delivery form of HCA is useful in the methods of the present invention such as exemplified below in Table 6.

TABLE 6
A controlled-delivery dosage form.

| Ingredient | | mg/Tablet | Percent |
|---|---|---|---|
| 1. | HCA magnesium salt | 500.00 mg | 71.43% |
| 2. | Microcrystalline cellulose | 17.00 mg | 2.42% |
| 3. | Dicalcium phosphate | 45.00 mg | 6.42% |
| 4. | Corn starch | 9.00 mg | 1.28% |
| 5. | TPGS | 46.00 mg | 6.60% |
| 6. | Hydrogenated vegetable oil | 50.00 mg | 7.14% |
| 7. | Cellulose acetate phthalate | 15.00 mg | 2.14% |
| 8. | Carbopol ® 974P Carbomer | 15.00 mg | 2.14% |
| 9. | Magnesium Sterate | 3.00 mg | 0.43% |

| | | |
|---|---|---|
| TOTAL | 700.00 mg | 100.00% |

1. Weigh and blend items 1-4 in a fluid bed dryer and blend for 4-5 minutes. Dissolve item #5 by heating to 40° C. until molten then stir with magnetic stir rod. After the powders are blended, continue steady blending while adding the TPGS as a molten liquid. Pour in all fluid until an even granulate is formed. Next melt the hydrogenated vegetable oil until molten and fluid in nature. Spray this material at the same time stirring with a magnetic stir rod. Continue blending with air at 30° C. When all the material is thoroughly coated and the granulate is hardened, spray the cellulose acetate phthalate that has been completely dissolved in ammoniated water. Continue spraying until all the granulate has been covered then allow to dry at room temperature in the fluid bed dryer with continuous blending. Remove the granulate from the bowl, when the granulate is dry, pass through an #093 screen using a D3 Fitzmill comminutor.

2. When the granulate has been dried and reduced in size, blend in fluid bed first with Carbopol_{—}974P, then when completely blended, add magnesium stearate and blend for 2-3 minutes.

3. Place the mixed granulate on a rotary press and compress the material into tablets with a weight of 700 mg and a fracture force of 10-15 kg.

### CONCLUSIONS

The invention provides that supplementation with HCA, its salts and related compounds are useful for increasing HDL levels and for improving not only HDL levels, but also for inhibiting the deleterious effects of a cholesterol challenge on the HDL:LDL ratio. This finding offers benefits against a range of cardiovascular diseases. These benefits of HCA are especially pronounced with the use of the preferred salts of the acid, potassium HCA and potassium-magnesium HCA, and may be further potentiated by the use of a controlled-release form of the compound. The discovery that HCA improves HDL levels and improves the HDL to LDL ratio in the face of dietary cholesterol challenge allows for the creation of novel and more efficacious approaches to preventing and ameliorating cardiovascular diseases. Furthermore, this discovery makes possible the development of adjuvant modalities that can be used to improve the results realized with other treatment compounds while at the same time reducing the side effects normally found with such drugs.

### EQUIVALENTS

From the foregoing detailed description of the invention, it should be apparent that unique methods for increasing the HDL:LDL ratio by administering HCA-containing compositions have been described resulting in improved therapeutic use. Although particular embodiments of the invention have been disclosed herein in detail, this has been done by way of example for purposes of illustration only, and is not intended to be limiting with respect to the scope of the appended claims which follow. In particular, it is contemplated by the inventor that substitutions, alterations, and modifications can be made to the invention without departing from the spirit and scope of the invention as defined by the claims. For instance, the choice of HCA salt, encapsulating agent, carrier or the choice of appropriate patient therapy based on these is believed to be matter of routine for a person of ordinary skill in the art with knowledge of the embodiments of the invention described herein.

What is claimed is:

1. A method for increasing serum high-density cholesterol lipoprotein (HDL) levels in an individual in need thereof comprising orally administering an effective amount of HCA.

2. The method of claim 1, wherein HCA is administered in a therapeutically-effective amount in its free acid or lactone form.

3. The method of claim 1, wherein HCA is supplied in a therapeutically-effective amount of the alkali metal salts potassium or sodium (−) HCA.

4. The method of claim 1, wherein HCA is supplied in a therapeutically-effective amount of the alkaline earth metal salts calcium or magnesium (−) HCA.

5. The method of claim 1, wherein HCA is supplied in a therapeutically-effective amount of a mixture the alkali metal salts and/or the alkaline earth metal salts of (−) HCA or some mixture of alkali metal salts and alkaline earth metal salts of (−) HCA or in the form of therapeutically effective amide and/or ester derivatives of HCA.

6. The method of claim 1, wherein HCA is supplied in a therapeutically-effective amount as the free acid, lactone or as one or more of the salts or other derivatives of the free acid and is delivered in a controlled release form.

7. A method for increasing the HDL:LDL ratio in a subject, comprising administering a therapeutically effective amount of a composition that is a salt of HCA and a suitable alkaline or alkali earth metal.

8. The method of claim 7, wherein the alkaline earth metal is calcium or magnesium.

9. The method of claim 7, wherein the alkali earth metal is potassium or sodium.

10. The method of claim 7, wherein the composition allows for a delivery dose of between about 750 mg and 10 grams.

11. The method of claim 1, wherein the composition is part of a controlled-release formulation.
